(19) United States
(12) Patent Application Publication
(10) Pub. No.: US 2004/0102615 A1
(43) Pub. Date: May 27, 2004

(54) Method for isolating and purifying a protein and resulting protein

(76) Inventors: Patrick Berna, Palaiseau (FR); Christelle Clement, Vic Le Comte (FR)

Correspondence Address: PALMER & DODGE, LLP, KATHLEEN M. WILLIAMS, 111 HUNTINGTON AVENUE, BOSTON, MA, 02199, US

(21) Appl. No.: 10/325,227

(22) Filed: Dec. 20, 2002

Related U.S. Application Data
(63) Continuation-in-part of application No. PCT/FR01/01985, filed on Jun. 22, 2001.

(30) Foreign Application Priority Data
Jun. 23, 2000 (FR) 00/08118

(51) Int. Cl. C07K 14/415

(52) U.S. Cl. 530/370

(57) ABSTRACT

The invention concerns a method for isolating and purifying a protein of interest, in particular from a complex medium such as a plant extract. Said method is characterized in that it compromises a step whereby a complex medium, comprising the solution containing the protein of interest to be purified and a solid support capable of enabling it absorption, is brought in the presence of an agent capable of causing said protein to precipitate in soluble form. The protein of interest is thus partly aggregated and absorbed on the solid support without substantial formation of macro-aggregates in the solution capable of spontaneous elutriation.

The present invention concerns a method for isolating and purifying a protein of interest in solution, comprising steps of partial aggregation and adsorption of said protein, notably from a complex medium that comprises lipids, and/or protein compounds and/or polysaccharides and/or pigments and/or polyphenols.

### BACKGROUND

Techniques for isolating and purifying proteins generally involve a step of solubilizing the protein to be isolated, followed by one or more successive steps seeking to separate the protein of interest, which is to be purified, from the initial medium.

The protein of interest can be separated from the medium in which it is solubilized by precipitation. In this procedure, the person skilled in the art can rely upon the addition of a protein-precipitating compound, which is adjusted to a concentration such that it permits the formation of molecular macroaggregates containing the protein, these molecular macroaggregates being molecular assemblies of a sufficient size to permit their spontaneous elutriation within the solution. As a general rule, the solid fraction comprising the molecular macroaggregates is separated from the soluble fraction by centrifuging, after which the insoluble pellet containing the protein of interest is recovered.

Such techniques for protein separation by precipitation are notably described in application DE 1,642,654, as well as in the patents U.S. Pat. No. 4,742,159; U.S. Pat. No. 5,169,936; U.S. Pat. No. 4,624,918; U.S. Pat. No. 4,470,969 and U.S. Pat. No. 4,343,735.

In particular, application DE 1,642,654 concerns the extraction of a lipase from Rhizopus arrhizus cell cultures. It is specified that the lipase can be separated by making it insoluble in organic solvents or in concentrated saline solutions, such as ammonium sulfate solutions.

Other separation methods for proteins comprise one or more filtration steps on various solid supports, such as filtrations seeking to retain most of the protein to be purified, essentially by mechanical action with size exclusion.

Among the filtration supports described in the prior art, diatomaceous earth has been used in methods for purifying proteins, such as the erythropoietin present in human urine or hepatitis B surface antigen (HbS) present in a bacterial lysate (see FR 2,467,214 and EP 0 480,525).

The protein of interest can also be isolated from the medium in which it is solubilized by passing this medium onto a chromatographic support, designed specifically to retain the protein of interest and thus to exclude the majority of undesirable material, such as unrelated proteins.

Among classically used chromatography methods, exclusion chromatography, ion-exchange chromatography and reversed-phase chromatography can be cited in particular.

The protein purification techniques of the prior art require isolating the desired protein in several steps distinct from one another by using very different, sometimes even incompatible, “tools” (such as, for example, obtaining an insoluble precipitate followed by chromatography).

These techniques sometimes permit obtaining the desired protein with a high degree of purity and with an economically satisfactory yield when these proteins are excreted in soluble form in media that is low in protein and/or the culture from secondary metabolites such as in certain natural fluids like urine or even medium from bacterial cultures or eukaryotic cell lines.

The purification of a protein of interest has sometimes proven to be more delicate and more difficult to implement when the initial medium contains a large number of secondary metabolites, in which the protein of interest is in the soluble form, or contains numerous undesirable proteins as well as lipids or polysaccharides, polyphenols or even certain pigments made up of aromatic structures bound to fatty chains, such as xanthophyll pigments.

This is particularly the case when the protein of interest must be purified from a plant material from a plant with a high lipid content, a high sugar content, or a high polyphenol content, as is the case for corn or tobacco.

The present invention effectively overcomes the disadvantages encountered in the prior art, particularly because the novel purification method does not include making the protein completely insoluble, leading to a spontaneously elutriating precipitate, but involves simply a step of partial aggregation and adsorption of the desired protein on a solid support.

This less-cumbersome method permits obtaining excellent results in the purification of proteins from complex media, particularly at industrial levels, since it can be conducted in a continuous manner, i.e. without interrupting the purification process, unlike techniques described in the prior art. Thus, the method of the invention has the advantage of permitting the purification of a protein of interest in a reduced number of steps. Moreover, this method permits working very quickly to purify molecules since it is adapted to the purification of proteins from so-called complex media comprising proteins, lipids, polysaccharides or other derivatives.

Moreover, it is observed that, according to the preferred method of the present invention, surprisingly and in a selective manner, approximately 97% of the undesired proteins, approximately 70% of the lipids and more than 99% of the starches present in the initial complex medium are eliminated.

### SUMMARY OF THE INVENTION

The subject of the invention is a method for isolating and purifying a protein of interest, comprising steps of partial aggregation and adsorption of said protein on a solid support, carried out in a simultaneous manner, said partial aggregation step comprising the introduction into said solution of a precipitating agent which generates molecular assemblies of said protein, which are of small size (microaggregates) and remain in suspension in the solution and are not able to spontaneously elutriate and which are adsorbed on said solid support. The method of the invention is advantageously used when purifying proteins of interest from a complex medium.

The isolation and purification method of the invention is characterized in that it comprises a step during which a complex medium, containing the protein of interest, which is to be purified, as well as a solid support able to permit its adsorption, is placed in the presence of an agent able to induce precipitation of this protein in solution, the protein of interest thus being precipitated and then adsorbed on the solid support without the substantial formation of macroaggregates in the solution.

The method according to the invention is particularly characterized in that the kinetics of the partial aggregation step are modified by the kinetics of the adsorption step in the sense that the adsorption kinetics promote the formation of microaggregates and oppose the formation of macroaggregates.

In another aspect, the invention relates to a protein of interest, characterized in that it is isolated and purified by the method described herein. In a preferred embodiment, the protein obtained by the implementation of the method disclosed herein is a recombinant protein, expressed in a multicellular animal, plant or fungal organism, or a virus.

Preferably, it is a recombinant protein expressed in a plant material, particularly an oleaginous, protein-containing plant material, or even a plant material rich in polysaccharides and/or polyphenols and/or pigments, notably fatty chain pigments.

The present invention also relates to a composition containing a protein of interest, resulting from the method, preferably a gastric lipase and in particular a dog gastric lipase, and most preferably a recombinant dog gastric lipase, characterized in that said composition is free of undesirable enzymes and/or enzymes responsible for side effects, in particular in that said composition is totally free of protease and amylase.

The present invention also concerns a pharmaceutical composition characterized in that it contains an extracted or recombinant gastric lipase, preferably a recombinant dog gastric lipase, such as described above, in combination with a pharmaceutically acceptable vehicle.

According to a particular embodiment of pharmaceutical compositions according to the invention, such compositions are designed for a daily administration of recombinant dog gastric lipase of 10,000 IU/kg to patients, or a daily administration of 4 g of recombinant dog gastric lipase.

Such pharmaceutical compositions can be presented as desired in the liquid, solid or powder form and also contain pharmaceutically acceptable vehicles well known to the person skilled in the art.

The invention also concerns the use of an extracted or recombinant dog gastric lipase, and preferably a recombinant dog gastric lipase, such as previously described for the manufacture of a medication designed for the treatment of pancreatic exocrine deficiencies that result particularly from chronic or acute pancreatitis, cystic fibrosis, pancreatic cancer or surgery of the pancreas as well as for treatment of malnutrition in the elderly or premature infants.

As used herein, the term “solid support” refers to a solid or semi-solid (e.g., a gel matrix) material to which a protein of interest binds upon microaggregation. A “solid support” as the term is used herein does not comprise (e.g., is not derivitized with) a specific ligand for the protein of interest.

As used herein, the term “specific ligand” is a moiety that preferentially binds a given protein of interest, to the substantial exclusion (e.g., greater than 10-fold higher affinity, preferably 100-fold higher or more, for the protein of interest) of other proteins. A classic example of a specific ligand is an antibody specific for a protein of interest. A specific ligand will have a discrete binding site for the protein of interest. Solid supports useful according to the methods of the invention need not have a specific ligand for the protein of interest.

As used herein, the term “partial aggregation” means that a protein of interest forms microaggregates but does not form macroaggregates upon addition of a given amount of a given precipitating agent. The term “microaggregates” refers to molecular assemblies containing the protein of interest to be purified, the size of which is sufficiently small for the molecular assemblies to remain in suspension in the solution. Microaggregates do not spontaneously elutriate. In contrast, “macroaggregates” are larger molecular assemblies of protein that spontaneously elutriate from solution. Thus, as the concentration of a precipitating agent or other precipitating influence increases, a protein of interest will first partially aggregate into microaggregates, and then, with increasing agent concentration or other precipitating influence (e.g., pH change), aggregate into macroaggregates.

As used herein, the term “precipitating agent” refers to a chemical agent that alter the hydration status of a protein in solution such that the protein becomes insoluble. A precipitating agent will cause a protein in solution to form aggregates (microaggregates or macroaggregates), the extent of which is dependent primarily upon the identity and concentration of the precipitating agent but also upon other factors, including, for example, the presence or absence of detergents, the pH of the solution, and the isoelectric point of the protein.

As used herein, the phrase “kinetics of partial aggregation are modified by the kinetics of adsorption” means that aggregates formed in the presence of a solid support tend to adsorb to the support while still microaggregates, rather than forming macroaggregates in solution. Thus, the kinetics of the aggregation are modified by the presence of the solid support, such that the formation of microaggregates is favored, and the formation of macroaggregates is opposed in the presence, relative to the absence of a solid support.

As used herein, the term “complex medium” refers to a medium comprising a protein of interest and one or more of: a protein other than the protein of interest; a lipid compound; a polysaccharide compound; a polyphenol; and a pigment.

As used herein, the term “substantial absence of precipitating agent” means that a solution either lacks any of a given precipitating agent, or comprises a concentration of such precipitating agent below the concentration at which microaggregates form. The concentration below the concentration at which microaggregates form is preferably well below such concentration, e.g., less than 50% of such concentration, preferably less than 20% or lower, e.g., less than 5% or less than 1% or preferably lower.

As used herein, the phrase “rich in polysaccharides and/or in polyphenols and/or in pigments” means that tissue of a given plant comprises one or more polysaccharides, polyphenols or pigments at a concentration that reduces the recovery of a protein of interest by 10% or more relative to the recovery of a similar protein from a milieu lacking such a polysaccharide, polyphenol or pigment when standard precipitation (i.e., macroaggregation) methods are used.

The present invention is illustrated, without being limited, by the following tables and figures:

Table 1: raw data for the effect of the quantity of ammonium sulfate on the adsorption on diatomaceous earth of recombinant gastric lipase expressed in corn.

Table 2: raw data for the effect of the quantity of sodium sulfate on the adsorption on diatomaceous earth of recombinant gastric lipase expressed in corn.

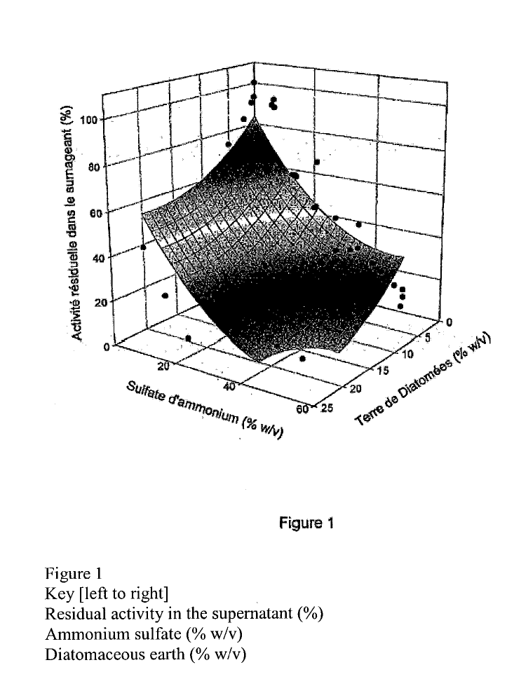

FIG. 1: effect of the quantity of ammonium sulfate on the adsorption on diatomaceous earth of recombinant gastric lipase expressed in corn.

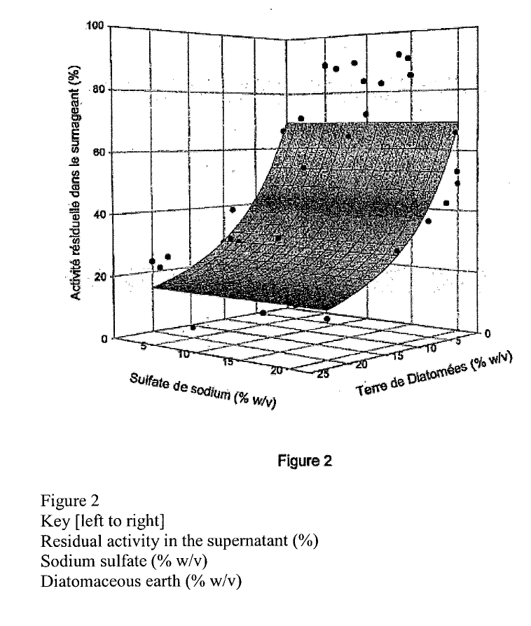

FIG. 2: effect of the quantity of sodium sulfate on the adsorption on diatomaceous earth of recombinant gastric lipase expressed in corn.

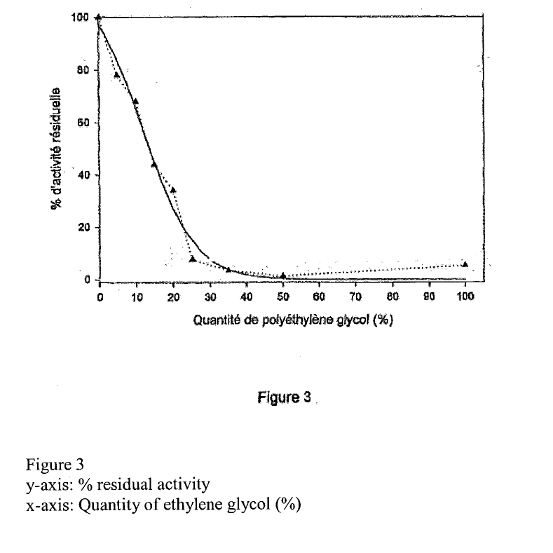

FIG. 3: effect of the quantity of polyethylene glycol (PEG 4000) on the adsorption on diatomaceous earth of recombinant gastric lipase expressed in corn.

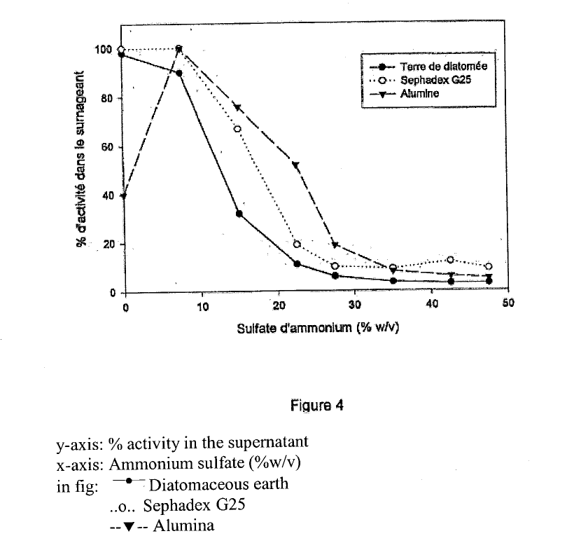

FIG. 4: effect of the type of solid support on the adsorption of recombinant gastric lipase in the presence of ammonium sulfate.

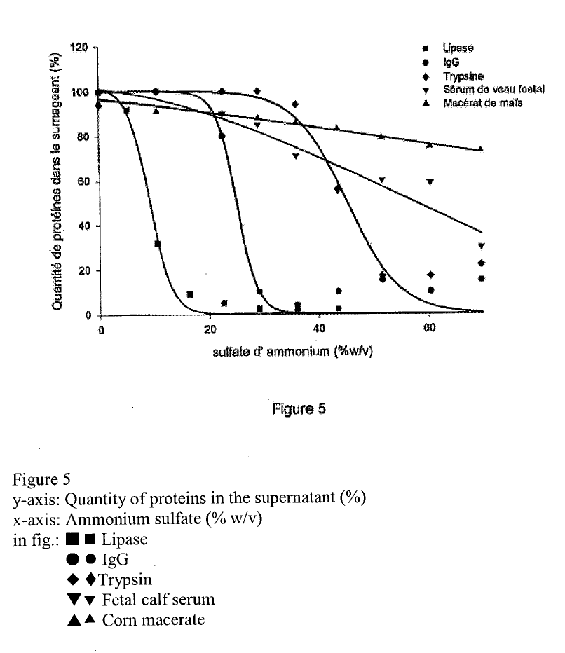

FIG. 5: effect of the quantity of ammonium sulfate on the adsorption of different types of proteins on diatomaceous earth.

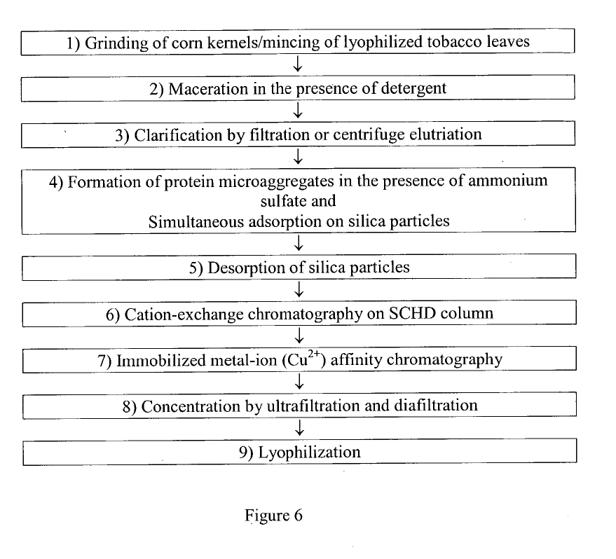

FIG. 6: general diagram for the purification of recombinant dog gastric lipase according to the method of the invention, from corn kernels or lyophilized tobacco leaves.

The successive steps of the method according to the invention are summarized in FIG. 6 and the corresponding legends are integrated in the present description.

### DETAILED DESCRIPTION OF THE INVENTION

The inventors have demonstrated a means for purifying a protein of interest in solution, notably from a complex medium, by the addition into the medium of a precipitating agent and a solid adsorbent support, thus permitting the adsorption of the protein to be purified directly onto the solid support while preventing the substantial formation of macroaggregates of the protein in solution.

The addition of the precipitating agent can be simultaneous with or can follow the introduction of the solid support after a brief or longer delay.

In complex media, and particularly media containing lipids and/or polysaccharides and/or polyphenols and/or fatty-chain pigments, the classical steps of purification by chromatography are rendered very ineffective. More particularly, the inventors have observed that in such solutions, the protein of interest, which is to be purified, is not selectively retained by the different chromatographic supports tested.

The absence of fixation of the protein, which is to be purified from a complex medium, on a chromatography support can be explained by two phenomena which may appear in conjunction:

on the one hand, undesirable compounds in the complex solution are adsorbed on the chromatographic support and thus saturate the majority of sites, which are then no longer accessible for the fixation of the protein of interest;

on the other hand, certain compounds, such as polysaccharides or lipids, are able to surround the protein molecules to be purified, and thus prevent any contact of the protein to be purified with the chromatographic support.

In aqueous solution, a solubilized protein is highly hydrated, i.e., ionic groups present at the protein surface attract and bind numerous water molecules by means of weak bonds (hydrogen bonds, Van der Waals attractions).

When a protein-precipitating agent, such as ammonium sulfate, is added to the solution containing the protein of interest, the ions of the precipitating agent attract water molecules and thus render them inaccessible to the protein initially in solution.

In the absence of a sufficient number of weak bonds between the protein molecules in solution and the neighboring water molecules, the protein molecules have a tendency to interact with one another and begin to aggregate. Classically, the concentration of precipitating agent is adjusted so that the protein molecule is largely free of weak bonds with the neighboring water molecules and so that the protein molecules initially in solution interact in such a way as to form macroaggregates having a sufficient size to permit their spontaneous elutriation in solution and their recovery, in general by centrifuging, in the form of a solid fraction found integrally in the pellet.

When the inventors attempted to separate a protein, dog gastric lipase, from a complex medium, in a plant extract, by means of ammonium sulfate, an unexpected phenomenon was observed. In fact, a triphasic solution was obtained after centrifuging the fraction rendered insoluble due to the addition of ammonium sulfate. This triphasic solution comprised a solid phase found in the pellet after centrifugation, and two liquid phases without distinct interface, the protein to be purified being found within the upper, lipophilic liquid phase. The absence of a distinct interface between the lower liquid phase and the upper [lipophilic] liquid phase made it difficult, if not impossible, to recover, with acceptable or satisfactory yields, the upper liquid fraction containing the protein to be purified. In any case, the recovery of the upper liquid phase containing the protein of interest involved a significant loss of the protein to be purified, and considerably reduced the economic utility of such a purification method.

The inventors therefore sought an effective and practical means for isolating a protein of interest in a complex medium.

The first aspect of the invention relates to a method for isolating and/or purifying a protein of interest in solution comprising steps of partial aggregation and adsorption of said protein on a solid support, said partial aggregation step comprising the introduction into said solution of a precipitating agent that generates molecular assemblies of said protein of small size, which cannot spontaneously elutriate and which are adsorbed on said solid support.

In contrast to the precipitation methods indicated above, the method of the invention comprises a step during which a precipitating agent is added to a medium comprising the solution containing the protein of interest as well as a solid support, preferably not derivitized with specific ligands that can interact with the protein of interest.

In this way, the protein of interest which begins to form molecular assemblies of small size (microaggregates) in the presence of the precipitating agent can immediately be adsorbed on the support. In particular, said support seems to favor the rapid discharge of the protein from the solution by adsorbing these protein microaggregates or even isolated molecules of the latter before the formation of a solid fraction that can be elutriated (macroaggregates).

Thus, the method of the invention is characterized in that the molecular assemblies of said protein are substantially in the form of protein aggregates of small size which remain in suspension in the solution (microaggregates).

Moreover, the partial aggregation and adsorption of said protein are simultaneous. Without wishing to be bound by any single mechanism, it is believed that the presence of the solid support in the solution containing the protein of interest at the same time that the precipitating agent modifies the reaction kinetics of the protein aggregation reaction, facilitating an immediate discharge of the protein molecules (microaggregates already formed and/or isolated molecules) from the solution by adsorption. This modification of the kinetics, which opposes the formation of large molecular assemblies (macroaggregates), therefore carries out the desired purification by means of a partial aggregation step.

The expression “microaggregates” refers to molecular assemblies containing the protein of interest to be purified, whose size is sufficiently small for the latter to remain in suspension in the solution. Thus, these molecular assemblies are not able to elutriate spontaneously. In contrast, “macroaggregates” refers to assemblies that spontaneously elutriate from solution.

The method of the invention has considerable advantages when compared with protein purification methods making use of a precipitation. On the one hand, it permits adsorbing the protein of interest on a support in a single step that can be integrated into an industrial process, and more particularly into a continuous industrial process. Moreover, the approach permits an optimal adjustment of the concentrations of precipitating agent added to the medium containing the protein to be purified, and more particularly a reduction in the quantity of precipitating agent necessary to “discharge” the protein of interest from the solution by adsorbing it on the solid support.

The proteins thus adsorbed can then be easily desorbed from the solid support according to classical techniques and in the absence of the precipitating agent, in order to be recovered and possibly subjected to other purification steps, for example, by chromatography.

Proteins of Interest that can be Purified by the Method of the Invention

The method according to the invention is particularly adapted to the purification of proteins from complex media, i.e., media containing, alone or in combination, protein compounds, i.e., proteins or parts of proteins not related to the protein of interest, polysaccharides, lipid compounds, and polyphenols and/or pigments, particularly fatty-chain pigments such as xanthophylls. Thus, the method of the invention can be implemented for the purification of proteins from material of animal, bacterial, viral or fungal origin and advantageously from biological material such as fetal serum, blood plasma or even from plant material, particularly from plant material rich in lipids, polysaccharides, polyphenols and/or fatty-chain pigments, such as oleaginous, protein-containing plants, plants with a high polysaccharide content, or even plants with a high pigment content.

In preferred embodiment, the method of the invention is used to purify a recombinant gastric lipase and in particular a recombinant dog gastric lipase expressed in a transgenic plant, such as corn, tobacco, tomato, canola, soy, rice, potato, carrot, wheat, barley, sunflower, lettuce or even oats.

In another preferred embodiment, recombinant dog gastric lipase is expressed in a transgenic plant, for example, as taught in patent application PCT FR 96/00606, published under the number WO 96/33277, the content of which is incorporated by reference in the present application.

More particularly, the recombinant dog gastric lipase, as it is isolated by the method described herein, is pure to at least 90% when referring to the area under the peak of a UV absorbance at 230 nm with respect to the total area under absorption peaks, preferably to at least 92% and in a most preferred embodiment to at least 95%.

The method of the present invention is not limited to the isolation of recombinant dog gastric lipase. Additionally, another aspect of the invention relates to a protein of interest purified by the method described herein. In addition to dog gastric lipase, several types of proteins of interest and, in particular, extracted or recombinant gastric lipases can in fact be separated from various complex media by means of this method with roughly equivalent degrees of purity. Some of the characteristics of the protein to be purified can be taken into account by the person skilled in the art to adjust the parameters of the method of the invention, such characteristics including molecular weight, surface properties, and isoelectric point.

a) Molecular Weight

The results of studies demonstrated that proteins of very different molecular weights can be isolated and purified by means of the method of the invention. By way of example, proteins of molecular weights between 20 and 200 kD could be separated by adsorption on diatomaceous earth after introducing an optimal concentration of ammonium sulfate into the solution where these proteins are found. The preliminary results obtained up until now nevertheless do not seem to indicate a directly proportional relationship between the molecular weight of the protein to be separated and the concentration of the precipitating agent.

b) Surface Properties and Isoelectric Point

The surface properties of the protein to be precipitated have a very dominant influence on its solubility. In fact, the easier it is to separate the water molecules bound to the surface of the protein, the easier it is to partially aggregate it. It is therefore important for the person skilled in the art to take into account the presence of hydrophobic and hydrophilic groups normally found on the surface of the protein of interest, when necessary, while conducting the purification according to the method of the invention. For example, the presence of hydrophilic groups on the surface of the protein will require overall a higher concentration of precipitating agent to partially aggregate it than if the protein has hydrophobic groups that confer upon it a lower basic solubility.

Another characteristic of the protein to be purified that should be considered by the person skilled in the art when adjusting the parameters of the method of the invention is the isoelectric point of the protein of interest. This characteristic also has an influence on the solubility of the protein in solution and should therefore be taken into account during the implementation of the method of the present invention.

The characteristics mentioned above permit an overall evaluation of the protein of interest, and the parameters of the method can then be adjusted more precisely and more rapidly as a function of the estimated solubility of the protein to be isolated.

Principal Parameters of the Method of the Invention

a) Precipitating Agents

According to the method of the present invention, several types of different precipitating agents can be used. The person skilled in the art can choose from among organic salts, inorganic salts, or even compounds of the polyalkylene glycol type and preferably polyethylene glycol. Ammonium acetate can be cited by way of example of organic salts, and for inorganic salts, ammonium sulfate or sodium sulfate can be cited. Cosmotropic salts as well as polyols, carbohydrates and compounds such as methylpentanediol (MPD) can also be used in the method according to the invention.

As shown in FIGS. 1, 2 and 3 of the present application, recombinant dog gastric lipase expressed in corn can be purified by using 3 different precipitating agents: ammonium sulfate, sodium sulfate; and polyethylene glycol. The precipitating agent most appropriate for the precipitation of the protein that one wishes to isolate can be chosen easily by the person skilled in the art.

With regard to the concentration of precipitating agent that is necessary for isolating a significant quantity of protein from the concerned medium, experiments show that this concentration varies as a function of the precipitating agent used. By way of example, the inventors used the method according to the invention in order to purify to homogeneity a recombinant dog gastric lipase from a plant material such as corn kernels or tobacco leaves. For the purification of this recombinant dog gastric lipase from a complex solution (a plant extract), ammonium sulfate concentrations between 10% and 60% by weight/volume, preferably between 15% and 45% by weight/volume and more preferably between 15% and 30% by weight/volume permitted recovering the largest concentration of lipase. On the other hand, the lipase concentrations are lowest when sodium sulfate is used. In this latter case, sodium sulfate concentrations vary preferably between 10% and 30% by weight/volume. When polyethylene glycol (PEG 4000) is used, optimal concentrations are between 20% and 40% and preferably between 25% and 35% by weight/volume.

The concentration of precipitating agent to be used is not only a function of its nature but also, to a certain extent, of the nature and the concentration of the adsorbent solid support used in the method of the invention.

b) Adsorbent Supports

Several types of solid supports can be used in order to adsorb the protein that one seeks to purify. Generally, the adsorption support is a solid support that is not derivitized with specific ligands able to interact with the protein of interest. Specific ligands should be understood as any ligand capable of establishing an affinity or hydrophobic bond, or electron donor-electron acceptor bond specifically with the desired protein. The solid support is therefore more particularly chosen from among organic or inorganic supports. By way of example of inorganic supports, the following can be cited: supports based on silica such as microporous glass or silica gel, or supports based on metal oxides, diatomaceous earth, alumina, perlites, as well as ceramics or zeolites. Among organic supports, the following can be cited by way of example: supports based on dextran, agarose, polyacrylamide, divinylbenzene polystyrene, methacrylate, nylon or cellulose. As illustrated in FIG. 4, several types of supports, notably very hydrophilic supports, can be used in an effective manner to adsorb recombinant gastric lipase in the presence of ammonium sulfate. The use of 3 different supports has been studied: diatomaceous earth, alumina and Sephadex G25 resin. As demonstrated in FIG. 4, each of these supports behaves essentially in the same way in gastric lipase adsorption experiments in the presence of ammonium sulfate.

A preferred solid support according to the invention is made up of diatomaceous earth. Used in the presence of ammonium sulfate, sodium sulfate or polyethylene glycol, diatomaceous earth permitted, in all 3 cases, the adsorption of almost all of the recombinant gastric lipase found in a complex solution.

With regard to the quantity of solid support necessary for the adsorption of an optimal concentration of the protein to be purified, this quantity can vary as a function of the nature of the solid support used. The person skilled in the art can easily determine the optimal quantity of solid support by conducting, for example, a preliminary standardization of the protein to be purified with various concentrations of solid supports.

More particularly, with regard to diatomaceous earth, optimal quantities permitting the purification of an acceptable concentration of protein found in solution are between 1% and 30% by weight/volume, more particularly between 1% and 3% by weight/volume. In a preferred embodiment, diatomaceous earth can be included in a filter of the frame type. The use of this type of filter can also permit the development of a continuous purification method.

In this context, an appropriate concentration of precipitating agent is introduced and dissolved in the complex medium (from which the protein of interest is purified) followed by the introduction of diatomaceous earth. The diatomaceous earth is held in suspension in the complex medium for a sufficient period to permit the maximal adsorption of protein. The suspension is then passed through a frame filter comprising, if necessary, a pre-layer of diatomaceous earth.

In the case of proteins that could not be isolated by adjusting the basic parameters of the method of the invention, particularly the choice and concentration of the precipitating agent and the solid support, a priori optional parameters including the use of particular reagents such as detergents, or adjustment of the pH should then be considered.

Even where the disclosed method works with a given precipitating agent and solid support, detergents and/or variations in pH can be utilized to optimize isolation conditions in a given complex medium or for a given protein of interest.

Additional Optional Parameters of the Method of the Invention

a) Use of Detergents

The use of detergents in the method of the present invention, although generally optional, can prove useful for modifying the selectivity of the method. In fact, the detergent can contribute to modifying the interactions between the molecules present in the complex medium and thus influence adsorption of certain desired proteins on the solid support. The use of several types of detergents is envisioned. By way of example, detergents of the Triton X-100 and Tween 20 type can be cited.

In a preferred embodiment of the method of the invention, when purifying dog gastric lipase from a complex medium, the protein is adsorbed on diatomaceous earth which is then rinsed with a glycine buffer (50 mM) at acidic pH in the presence of a detergent. The detergent preferred in this embodiment of the invention is a non-ionic detergent, for example Triton X-100, BRIJ 35 or similar detergents.

b) Influence of pH

pH is a parameter that can be important for the implementation of certain preferred embodiments of the method of the invention. It is possible to modify the pH of the complex medium from which the protein of interest is isolated in order to modify the solubility conditions of this protein in solution.

The range of pH values used within the scope of the method of the present invention is considerable and can generally vary from 2 to 10. By way of example, recombinant gastric lipase was isolated at pH 3, and trypsin and IgGs were isolated at pH 7. The same pH ranges can also be envisioned in the case of complex mixtures from which proteins must be isolated. By way of example, proteins found in fetal calf serum were isolated at pH 7 (see FIG. 5). In contrast, proteins found in a corn macerate were isolated at pH 3.

In a more general embodiment, the person skilled in the art can readily design and perform preliminary tests in which the critical parameters (type and concentration of precipitating agent, solid support) and the optional parameters (addition of additional reagents, pH) of the method are varied one by one until optimal conditions are reached.

The method according to the invention also relates to a desorption step for the protein of interest from the solid support. This step occurs in the absence of precipitating agent.

After rinsing, the adsorbed protein is desorbed by elution at acidic pH in a buffer not containing the precipitating agent. The entire desorption eluate is recovered.

In one particular embodiment of the invention, the desorption solution is then optionally subjected to a filtration step. The filtering membrane preferably has a retention threshold between 5 and 40 micrometers, and in a most preferred manner, a retention threshold of 10 micrometers.

Once the adsorption and desorption steps have been conducted, the eluate from the desorption step, or the filtrate from the optional fine filtration step, is preferably, but not necessarily, subjected to one or more final purification steps, in order to obtain a purified final product. Some of the different final purification steps that can be used by the person skilled in the art are described below.

Optional Final Purification Steps

These final purification steps are chosen by the person skilled in the art as a function of the nature of the protein of interest to be purified (positively or negatively charged, hydrophilic or hydrophobic, rich in histidine, etc.)

a) Chromatography

The eluate from the desorption step, or the filtrate from the optional fine filtration step, for example, can be subjected to one or more chromatographic steps such as ion-exchange chromatography, size-exclusion chromatography, hydrophobic interaction chromatography, immobilized metal-ion affinity chromatography, affinity chromatography or high performance liquid chromatography (HPLC). This step can alternatively comprise a step of passage of the protein of interest desorbed from the solid support onto a chromatographic support for cation exchange.

By way of particular example according to the invention, for the purification of recombinant dog gastric lipase, the desorption solution is first diluted if necessary in order to reduce the ionic strength and thus promote the adsorption of the protein of interest on an ion exchanger. The solution is then optionally subjected to a fine filtration step, and then loaded onto a cation-exchange chromatographic column (e.g., S-ceramic Hyper D type sold by BIOSEPRA). The loaded column successively undergoes two washings: a first washing in a glycine buffer (50 mM) at pH 3 in the presence of a detergent (Triton X-100, 1 mM), in order to eliminate lipids which were possibly adsorbed on the column, and a second washing in a glycine buffer (50 mM) at pH 3, in order to eliminate the residual detergent. The column is finally eluted by means of an acetate/acetic acid buffer (50 mM) at pH 4, since these particular conditions permit directly loading the resulting eluate onto another chromatographic column, without a preliminary dialysis or dialysis.

The eluate emerging from the cation-exchange chromatographic column can then be loaded onto an immobilized metal-ion affinity resin chromatographic (IMAC) column. In a preferred embodiment, the chromatographic gel is filled with copper (Cu II). The column is subjected to a washing with a buffer identical to the loading buffer, then eluted by means of a glycine buffer (10 mM) in the presence of ammonium acetate at appropriate pH.

In the particular example of the purification of dog gastric lipase, which is made up of a glycoprotein of 49 kDa containing 13% carbohydrates and bears 14 histidine residues, the histidines were exploited during one particular chromatographic step of the method of the invention. Histidine residues exposed on the surface of this protein have the property of being bound by means of coordination bonds to certain metal ions immobilized on chromatographic supports, such as nickel (Ni II) or copper (Cu II) ions. Such chromatographic supports comprising immobilized metal ions are, for example, columns of the IMAC type mentioned above and used in Examples 1 and 2.

Thus, for the purification of dog gastric lipase, elution on the IMAC chromatographic column was performed by means of the glycine buffer and in the presence of ammonium acetate at pH 3. The presence of sodium acetate in the elution buffer is advantageous in that the enzymatic activity of the purified recombinant gastric lipase is less affected in the presence of this volatile compound than in the presence of a classical sodium chloride buffer due to a lower saline concentration, and, moreover, constitutes more favorable conditions for a final lyophilization of the eluate containing the recombinant dog gastric lipase.

The elution was conducted at a strongly acidic pH, which is probably due to an unusually low pKa of less than 4 at the site of interaction of the recombinant gastric lipase with the support.

b) Filtration

The protein thus purified can then be subjected to one or more additional ultrafiltration and/or dialysis steps, notably in order to concentrate the protein solution, but also to place the protein of interest under the physicochemical conditions of stability necessary for a final lyophilization step.

The ultrafiltration and/or dialysis step can be frontal, tangential or helical, as desired.

In a particular embodiment of the method according to the invention, a frontal ultrafiltration step is carried out on a membrane of the polyether sulfone type with a cut-off threshold of 30 kDa, which permits concentrating the eluate by a factor of at least 10. The resulting filtrate is then subjected to two dialysis steps permitting a dilution by a factor of 100, of the saline concentration of the filtrate and an adjustment of the pH to a value compatible with an absence of denaturation of the purified recombinant protein.

The ultrafiltered and then dialyzed solution can then also undergo an additional filtration step designed to eliminate bacteria possibly present, the filter here having an average pore diameter of 0.22 μm.

The method according to the invention can also comprise a drying step for the purified protein solution. This drying step can thus be realized particularly by lyophilization or atomization of the purified protein according to techniques well known to the person skilled in the art.

Purification of Proteins Expressed in Plants (Preliminary Steps)

The purification method according to the invention will advantageously be used to isolate a protein of interest produced in plants such as corn, tobacco, tomato, canola, soy, rice, potato or even carrot.

In particular, the method of the invention is suited to the purification of recombinant proteins expressed in plants. Thus, the invention also pertains to a method characterized in that it comprises a first step of grinding kernels or mincing leaves, followed by a step of clarification by filtration or centrifugation.

The preliminary principal steps that can be envisioned before implementation of the method according to the invention are described below.

a) Extraction

In a preferred manner, the purification method according to the invention can comprise a first step consisting of extracting most of the proteins from the crude plant material, particularly a plant material from a transgenic plant expressing the recombinant protein of interest.

In the case of corn, protein is extracted from a homogenate obtained from kernels ground on screens with a diameter of 1 to 3 mm.

In the case of extraction from tobacco leaves, the leaves are optionally lyophilized, then ground until a powder is obtained.

b) Maceration

The powder from this first processing of the plant material can be macerated in an acidic buffer in the presence of detergent, the buffer optionally being supplemented with a chelating agent such as EDTA.

Any type of detergent can be used during the maceration step, so as to solubilize most of the protein of interest present in the initial homogenate, in particular the plant material powder described above.

Advantageously, a non-ionic detergent will be used, i.e., a detergent that cannot be bound on chromatography supports during possible final purification phases of the product of interest.

Therefore detergents such as Triton X-100 or BRIJ 35 will be preferred, preferably used at a concentration equal to 10 times the critical micelle concentration (CMC).

Consequently, Triton X-100 will preferably be used at a concentration comprised between 0.5 mM and 2 mM, preferably at a concentration of 1 mM, during the maceration step.

The maceration step has a duration between 5 and 20 hours, and is preferably approximately 15 hours, for example, a duration of 16 hours. During the maceration step, the pH is advantageously set between 2.5 and 4, and is preferably adjusted to 3.

c) Clarification

The maceration step can be followed by a clarification step designed to eliminate large insoluble particles, such as debris from the initial plant material, aggregates, etc. Clarification can be conducted by any technique well known to the person skilled in the art.

Preferably, clarification by filtering, elutriating or centrifuging will be used.

In the case where clarification is carried out by centrifuging, advantageously the crude extract will be centrifuged at between about 8000 and 15,000×g, preferably 10,000×g and for a time between about 3 and about 10 min., and in a most preferred manner, for about 5 minutes.

Once these preliminary steps are completed, the protein of interest is isolated from the supernatant fraction of the extract by the application of the principal parameters of the method of the invention.

### EXAMPLES

### Example 1

### Purification Method for Recombinant Dog Gastric Lipase from Corn Kernels

A) Reagents

1) Chemical Products

The different chemical products used in the purification method of recombinant dog gastric lipase (psl rDGL) are at least of analytical quality. A list of these products is detailed below:

| NAME | SUPPLIER | REFERENCE | REMARKS |
|---|---|---|---|
| Tributyrin | Fluka | 91012 | |
| Bovine serum | Sigma | A-7906 | |
| albumin | | | |
| Taurodeoxycholic | Sigma | T-0875 | sodium salt |
| acid | | | (NaTDC) |
| NaCl | Merck | 10604,1000 | |
| NaOH | Merck | 9142.0500 | |
| Bicinchoninic acid | Sigma | B-9643 | |
| Copper sulfate | Sigma | C-2284 | |
| Bovine serum | Sigma | P-0914 | standard |
| albumin (BSA) | | | solution |
| Glycine | Merck | 4201.1000 | |
| HCl | Merck | 100.317 | fuming |
| EDTA | Sigma | E-5134 | disodium salt |
| Triton X-100 | Sigma | X-100 | |
| DICB (diatomaceous | Meristem | | |
| earth) | Therapeutics | | |
| Ammonium sulfate | Sigma | A-5132 | |
| Magnesium chloride | Sigma | M-8266 | anhydrous |
| Sodium acetate | Merck | 106268 | anhydrous |
| Ammonium acetate | Sigma | A-7330 | |
| Acetic acid | Merck | 1.00062 | glacial |
| NaOH | Merck | 1.05587.2500 | |
| Mannitol | Sigma | M-9647 | |

2) Buffer Solutions

The buffers used are listed in the following table:

| | | |
|---|---|---|
| Maceration | 50 mM Glycine-HCl, pH 2.5 | 14 volumes referred to the |
| | 250 mM Sodium chloride | weight of the meal used |
| | 1 mM Triton X- 100 | |
| | 1 mM EDTA | |
| Clarcel washing | 50 mM Glycine-HCl, pH 2.5 | 7 volumes referred to the |
| | 40% Ammonium sulfate (0.229 | weight of the diatomaceous |
| | Kg/L) | earth |
| | 1 mM Triton X-100 | |
| | 75 mM Magnesium chloride | |
| Clarcel desorption | 50 mM Glycine-HCl, pH 2.5 | 17 volumes referred to the |
| | 1 mM Triton X-100 | weight of the diatomaceous |
| | 75 mM Magnesium chloride | earth |
| Dilution of the retained | 50 mM Glycine-HCl, pH 3.0 | Qs sufficient for adequate |
| material | | conductivity |
| Concentrated equilibration | 500 mM Glycine-HCl, pH 3.0 | 7 column volumes |
| 10X SCHD | | |
| SCHD equilibration | 50 mM Glycine-HCl, pH 3.0 | 7 column volumes |
| | 50 mM Sodium chloride | |
| | 1 mM Triton X-100 | |
| | 75 mM Magnesium chloride | |
| Washing 1, SCHD | SCHD equilibration | 17 column volumes |
| Washing 2, SCHD | 50 mM Glycine-HCl, pH 3.0 | 16 column volumes |
| | 50 mM Sodium chloride | |
| | 75 mM Magnesium chloride | |
| Elution, SCHD | 50 mM Sodium acetate, pH 4 | 8 column volumes |
| | 50 mM Acetic acid | |
| | 500 mM Sodium chloride | |
| Regeneration solutions, | 1 M NaCl | 6 column volumes |
| SCHD | 500 nM NaOH | 6 column volumes |
| Equilibration, IMAC | 50 mM Sodium acetate | 11 column volumes |
| | pH 4.0 | |
| | 50 mM Acetic acid | |
| | 500 mM NaCl | |
| Washing 1, IMAC | IMAC equilibration | 16 column volumes |
| Copper sulfate | 50 mM copper sulfate in water | 3.5 column volumes |
| | subjected to osmosis | |
| Washing 2, IMAC | 10 mM Glycine-HCl, pH 3.5 | 33 column volumes |
| | 500 mM Ammonium acetate | |
| Elution, IMAC | 10 mM Glycine-HCl, pH 2.8 | 7 column volumes |
| | 1 M Ammonium acetate | |
| Regeneration solutions, | 1 M HCl | 5 column volumes |
| IMAC | 500 mM NaOH | 5 column volumes |
| Dialysis | 20 mM Citric acid-NaOH, | |
| | pH 4.0 | |

B) Protein Measurement

The concentration of proteins is determined by means of bicinchoninic acid (BCA) (Smith et al., Anal. Biochem. (1985), 150,76-85). The reference protein is a control solution of 1 mg/ml of BSA, from Sigma. The absorbance is measured on a 96-well microplate with an IEMS/MF reader sold by Labsystem, equipped with a 540-nm interference filter. Number analyzed per lot: 4.

C) Measurement of Activity of the Recombinant Gastric Lipase

Lipase activity is measured by titrimetry with a Mettler brand DL25 titrimeter or a Metrohm Titrino brand 718 titrimeter, at pH 5.0 and at 37° C. on tributyrin (Gargouri et al. Gastroenterology (1986), 91: 919-925). Number of analyses per lot: 4.

D) Monitoring the Purification Method

The method is monitored at different steps by measurement of the activity on tributryrin, measurement of the protein concentration by BCA and estimation of the percentage of purity by reversed-phase chromatographic analysis on a C4 column (VYDAC, column C4, 300 angstroms, 250 mm×4 mm)

During the steps of purification by chromatography, proteins are detected at 280 nm.

E) Grinding

700 kg of corn are ground with a Forplex impact grinder

The grinder is cooled with a liquid nitrogen current. Thus the temperature of the meal does not surpass 20° C.

F) Maceration

In a first operation, the optimal conditions used are the following:

Maceration 1:

Plant/buffer ratio: 1+8 (8 liters of buffer per 1 kg of meal)

Duration of maceration: 10 h

Temperature: 20° C.

Maceration 2:

Plant/buffer ratio: 1+2

Duration of maceration 2 h

Temperature 20±2° C.

Maceration 3:

Plant/buffer ratio: 1+4

Duration of maceration: 8 h

Temperature: 20±2° C.

Operating Method:

The meal is introduced manually into a stainless steel vat of 10,800 liters, that has first been filled with a maceration buffer at pH 2.5.

The mixture is stirred. The pH of macerate 1 should then be 3.0±0.1: if it is not, it is necessary to adjust this pH with hydrochloric acid.

The slurries obtained after the first and second elutriations are returned to the macerator.

The maceration buffer volume added for macerations 2 and 3 is calculated from the quantity of meal used initially.

In a variant of the method described above, it is also possible to proceed in the following manner, which permits reducing the time involved in this step while maintaining the lipase extraction level.

Operating Method:

Maceration 1: 5 h, then elutriation 1 (duration approximately 15 h)

As soon as slurries arrive from elutriation 1: proceed to maceration 2.

Maceration 2: 2 h (counted from the end of elutriation 1), then elutriation 2 (duration approximately 6 h).

As soon as slurries arrive from elutriation 2: proceed to maceration 3.

Maceration 3: 8 h (counted from the end of elutriation 2), then elutriation 3 (duration approximately 10 h).

G) Elutriation

Operating Method:

After each maceration, the macerated material obtained is passed by means of a lobe pump into a centrifuge elutriating device whose average flow rate is 400 liters/h.

The crude extracts are collected in a stainless steel vat. They are stored at approximately 4° C. Crude extract 2 is mixed with crude extract 1. In addition, crude extract 3 is mixed with crude extracts 1 and 2 in order to form the final crude extract stirred in a stainless steel vat of 10,800 liters.

H) Prepurification

1) Treatment with Ammonium Sulfate and Adsorption on Diatomaceous Earth

Operating Method:

Then add 0.164 Kg/L of ammonium sulfate after dissolving 1.5% (w/v) diatomaceous earth (Clarcel DIC B, by weight of Clarcel referred to the volume of crude extract).

The mixture is stirred for 30±5 minutes in a vat at ambient temperature.

2) Filtration, Washing and Desorption on Diatomaceous Earth

Operating Method:

Filtering

After adsorption, stirring is stopped for 1 hour.

After adsorption, the suspension is filtered on a filter already containing a prelayer made with 10 kg of Clarcel

The filtrate obtained (not containing lipase) is eliminated.

Washing

The filter cake is then washed by using approximately 7 volumes of washing buffer for 1 kg of Clarcel having served for accretion.

The Clarcel cake is then dried.

Desorption

Desorption is conducted with approximately 17 volumes of desorption buffer per kg of Clarcel used for accretion.

The Clarcel cake is then resuspended by activating the stirring of the monoplate filter. After 30 minutes of stirring, the suspension is filtered.

The filtrate obtained is collected.

I) Filtration

Two types of filtration are tested.

1) Filtration

Operating Method:

The desorbed fraction is filtered on 40×40 cm K300 plates (cellulose plates containing Kieselguhr [diatomaceous earth] and perlite) from Seitz, of average pore size of 10 μm.

The filtrate obtained is collected in a vat.

According to a variant of this step, one can proceed as described below, which permits reducing technical problems such as clogging, opacity of the fractions, duration of operations, etc.

2) Desporption/Filtration

Operating Method:

Resuspend the Clarcel DIC B with the desorption buffer (17 volumes/weight of Clarcel), and stir for 30±5 minutes

Prepare the multiplate filter by equipping it with K300 filters.

Filter the Clarcel in suspension on this multiplate filter. At the end of filtration, force through 2 volumes of desorption buffer.

Recover the solution.

J) Ultrafiltration:

Operating Method

The filtrate is concentrated by means of an ultrafiltration system, which is equipped with cartridges of polysulfone whose membrane cut-off threshold is 30 kd.

The retained material is concentrated approximately four times.

K) Dilution

The retained material stored at approximately 4° C. is homogenized and diluted with a dilution buffer so as to obtain a conductivity equal to or greater by +1 mS than that of the SCHD equilibration buffer.

L) SCHD Chromatography

SCHD chromatography corresponds to a cation-exchange chromatography, S-Ceramic-HyperD (Biosepra). The matrix of this resin is made up of ceramic silica and dextran onto which sulfonate groups are grafted; the particle size of the beads is 60 μm.

Chromatography is conducted at ambient temperature.

M) IMAC Chromatography

IMAC chromatography is conducted on a resin whose matrix is made up of polymethacrylate onto which iminodiacetic acid residues are grafted (650M EMD Chelate fractogel, MERCK). These groups promote the binding of metal ions, particularly copper, which bind the lipase in turn by means of their free coordination sites.

The particle size of the beads is 40 to 90 μm.

Chromatography is conducted at ambient temperature.

N) Dialysis

The fraction obtained is immediately diluted with two volumes of dialysis buffer per volume fraction, then is concentrated by means of a Millipore HUF/50 ultrafiltration system equipped with polyether sulfone cartridges until a protein concentration between 6 and 7 mg/ml is obtained. The membrane cut-off threshold is 30 kd. The temperature is between 17° and 19° C.

A dialysis is then conducted with a constant protein concentration until a conductivity and pH identical to those of the dialysis buffer are obtained.

O) Lyophilization

The dialyzed product is then lyophilized in bulk or in flasks according to the method below:

After distributing the solution into aliquot fractions of 1 ml in flasks containing 5 ml of Wheaton serum, plugs (gray butyl) are positioned on the flasks without sealing them and the flasks are positioned in plugged or corked boxes. The plugged or corked boxes containing the flasks are introduced into the lyophilizer whose racks have been stabilized beforehand at a temperature of −45° C. This freezing phase is continued for 3 h at atmospheric pressure. The pressure is then reduced to 0.15 mbar, then the temperature of the racks is raised to +30° C. with a return setting to full vacuum (1 μbar) for a temperature of −10° C. The cyclic energy metering device is positioned at 5%, which results in a temperature rise rate of 5° C. per hour. Between 62 and 64 hours after the beginning of lyophilization, the temperature of the samples is stable and allows terminating the lyophilization operation. The vacuum is then broken by nitrogen C filtered through a filter of mean pore diameter of 0.2 μm and the flasks are stoppered and sealed with aluminum caps.

### Example 2

### Purification Method for Recombinant Dog Gastric Lipase from Transgenic Tobacco Leaves

The materials, chemical products, buffer solutions, filters and chromatographic supports are identical to those described for Example 1

A) Grinding of Leaves, Clarification and Adsorption on Diatomaceous Earth in the Presence of Ammonium Sulfate

The lyophilized leaves are ground by means of a Waring blender until a powder is obtained.

The powder is macerated for 15 min with slow stirring in 0.2 M NaCl and the pH is maintained at 3 with 1 M HCl (30 ml of NaCl/g of dry weight).

The homogenized product is centrifuged at 10,000 g at +4° C. for 15 min. The supernatant obtained is filtered on Miracloth.

The homogenized product is filtered and contacted with diatomaceous earth, then with 40% ammonium sulfate for saturation. Stirring is continued for 45 min.

The mixture is filtered on 20-μm 3 Chr Whatman filter paper and the remaining cake is taken up in a 10 mM glycine/HCl-0.2 M NaCl-40% ammonium sulfate washing buffer, pH 3 (1/5 volume HF+) and stirred for 5 min.

The homogenized product is filtered on 3 Chr 20-μm Whatman filter paper and the cake is recovered in a 10 mM glycine/HCl pH 3-0.2 M NaCl elution buffer (1 g of initial earth/15 ml of elution buffer. The mixture is stirred for 5 min, then filtered on 20-μm 3 Chr Whatman filter paper.

B) Cation-Exchanger Resin Chromatography

The filtrate from step A) is filtered on a 0.45 μm filter. The sample is then diluted with citrate-phosphate buffer, pH 3 (approximately 1/9) to permit adjusting the conductivity to that of the equilibration buffer of the column.

Chromatography takes place at 5° C. under the conditions described in the following table.

Protocol for cation-exchanger resin chromatography

| | | Flow rate | Number of column |
|---|---|---|---|
| Steps | Solution | (ml/min) | volumes |
| | 20 mM citrate | 2 | 10 |
| | phosphate- pH 3 | | |
| | 50 mM NaCl buffer | | |
| Loading | diluted F2 | 2 | |
| Washing | 20 mM citrate- | 2 | 40 |
| | phosphate buffer, | | |
| | pH 5.5 | | |
| | Triethanolamine | 2 | 15 |
| | buffer | | |
| Elution | 20 mM, pH 7 | 2 | 15 |

Citrate-phosphate buffers are citric acid+dibasic sodium phosphate.

Equilibration buffer: 35% phosphate and 63% citric acid.

Washing buffer: 63% phosphate and 28% citric acid.

C) IMAC-Cu II Resin Chromatography

This step is identical to step M) of Example 1.

The ultrafiltration, dialysis and lyophilization steps are identical to steps N) and O) described in Example 1.

TABLE 1

| Ammonium sulfate | Residual activity | Diatomaceous earth |
|---|---|---|
| (% v/w) | (%) | (% w/v) |
| 0.00 | 100.28 | 0.00 |
| 0.00 | 93.28 | 0.10 |
| 0.00 | 90.76 | 0.50 |
| 0.00 | 83.75 | 2.00 |
| 0.00 | 73.67 | 5.00 |
| 0.00 | 57.42 | 10.00 |
| 0.00 | 38.66 | 20.00 |
| 6.97 | 89.36 | 0.00 |
| 6.97 | 93.28 | 0.10 |
| 6.97 | 91.04 | 0.50 |
| 6.97 | 45.94 | 2.00 |
| 6.97 | 26.05 | 5.00 |
| 6.97 | 25.27 | 10.00 |
| 6.97 | 18.49 | 20.00 |
| 13.94 | 56.86 | 0.00 |
| 13.94 | 57.70 | 0.10 |
| 13.94 | 58.26 | 0.50 |
| 13.94 | 29.69 | 2.00 |
| 13.94 | 36.41 | 5.00 |
| 13.94 | 19.33 | 10.00 |
| 13.94 | 1.40 | 20.00 |
| 20.91 | 65.83 | 0.00 |
| 20.91 | 43.70 | 0.10 |
| 20.91 | 43.70 | 0.50 |
| 20.91 | 33.33 | 2.00 |
| 20.91 | 16.25 | 5.00 |
| 20.91 | 24.93 | 10.00 |
| 20.91 | 17.65 | 20.00 |
| 27.88 | 23.81 | 0.00 |
| 27.88 | 32.49 | 0.10 |
| 27.88 | 40.34 | 0.50 |
| 27.88 | 12.61 | 2.00 |
| 27.88 | 14.01 | 5.00 |
| 27.88 | 22.13 | 10.00 |
| 27.88 | 22.41 | 20.00 |
| 34.85 | 8.96 | 0.00 |
| 34.85 | 38.66 | 0.10 |
| 34.85 | 22.69 | 0.50 |
| 34.85 | 28.29 | 2.00 |
| 34.85 | 31.37 | 5.00 |
| 34.85 | 20.17 | 10.00 |
| 34.85 | 14.85 | 20.00 |
| 41.82 | 14.01 | 0.00 |
| 41.82 | 19.89 | 0.10 |
| 41.82 | 24.93 | 0.50 |
| 41.82 | 24.09 | 2.00 |
| 41.82 | 34.73 | 5.00 |
| 41.82 | 28.29 | 10.00 |
| 41.82 | 9.80 | 20.00 |
| 48.80 | 7.84 | 0.00 |
| 48.80 | 11.48 | 0.10 |
| 48.80 | 3.64 | 0.50 |
| 48.80 | 15.97 | 2.00 |
| 48.80 | 26.05 | 5.00 |
| 48.80 | 6.44 | 10.00 |
| 48.80 | 7.28 | 20.00 |

TABLE 2

| Sodium sulfate | Residual activity | Diatomaceous earth |
|---|---|---|
| (% v/w) | (%) | (% w/v) |
| 0.00 | 100.13 | 0.00 |
| 0.00 | 100.26 | 0.10 |
| 0.00 | 66.41 | 0.50 |
| 0.00 | 48.51 | 2.00 |
| 0.00 | 37.74 | 5.00 |
| 0.00 | 27.37 | 10.00 |
| 0.00 | 23.35 | 20.00 |
| 1.00 | 100.78 | 0.00 |
| 1.00 | 66.54 | 0.10 |
| 1.00 | 51.88 | 0.50 |
| 1.00 | 42.80 | 2.00 |
| 1.00 | 39.17 | 5.00 |
| 1.00 | 28.79 | 10.00 |
| 1.00 | 21.53 | 20.00 |
| 2.00 | 63.81 | 0.00 |
| 2.00 | 71.08 | 0.10 |
| 2.00 | 51.36 | 0.50 |
| 2.00 | 28.96 | 2.00 |
| 2.00 | 33.72 | 5.00 |
| 2.00 | 27.89 | 10.00 |
| 2.00 | 25.42 | 20.00 |
| 5.00 | 90.01 | 0.00 |
| 5.00 | 4.80 | 0.10 |
| 5.00 | 5.45 | 0.50 |
| 5.00 | 4.54 | 2.00 |
| 5.00 | 4.93 | 5.00 |
| 5.00 | 4.02 | 10.00 |
| 5.00 | 3.50 | 20.00 |
| 10.00 | 65.11 | 0.00 |
| 10.00 | 72.37 | 0.10 |
| 10.00 | 83.79 | 0.50 |
| 10.00 | 89.88 | 2.00 |
| 10.00 | 87.55 | 5.00 |
| 10.00 | 54.47 | 10.00 |
| 10.00 | 42.15 | 20.00 |
| 15.00 | 57.85 | 0.00 |
| 15.00 | 85.34 | 0.10 |
| 15.00 | 90.92 | 0.50 |
| 15.00 | 91.96 | 2.00 |
| 15.00 | 82.10 | 5.00 |
| 15.00 | 64.85 | 10.00 |
| 15.00 | 34.24 | 20.00 |
| 20.00 | 49.16 | 0.00 |
| 20.00 | 53.18 | 0.10 |
| 20.00 | 66.02 | 0.50 |
| 20.00 | 42.80 | 2.00 |
| 20.00 | 37.48 | 5.00 |
| 20.00 | 29.18 | 10.00 |
| 20.00 | 12.06 | 20.00 |

1. A method for isolating or purifying a protein of interest from a solution, the method comprising the steps of:
a) partial aggregation of said protein; and
b) adsorption of said protein on a solid support,
said partial aggregation step comprising the introduction into said solution of a precipitating agent which generates molecular assemblies of said protein which cannot spontaneously elutriate, wherein said molecular assemblies adsorb on said solid support.

2. The method according to claim 1, wherein the molecular assemblies of said protein are substantially formed of protein microaggregates that remain in suspension in the solution.

3. The method according to claim 1, wherein the partial aggregation and adsorption of said protein are simultaneous.

4. The method according to claim 1, wherein the kinetics of the partial aggregation step are modified by the kinetics of the adsorption step.

5. The method of claim 4, wherein the adsorption kinetics promote the formation of microaggregates and oppose the formation of macroaggregates.

6. The method according to claim 1, wherein the protein of interest is isolated or purified from a complex medium.

7. The method according to claim 6, wherein the medium contains said protein and one or more compounds chosen from among a protein other than said protein of interest, lipid compound, a polysaccharide compound, a polyphenol and a pigment.

8. The method according to claim 1, further characterized in that the precipitating agent is added to the solution containing said protein simultaneously with or after the introduction of said solid support.

9. The method according to claim 8, further characterized in that the precipitating agent is polyalkylene glycol, a polyol, a sugar, or an organic or inorganic precipitating salt.

10. The method according to claim 9, further characterized in that the polyalkylene glycol compound is a polyethylene glycol.

11. The method according to claim 9, further characterized in that the organic precipitating salt is ammonium acetate.

12. The method according to claim 9, further characterized in that the inorganic precipitating salt is ammonium sulfate or sodium sulfate.

13. The method according to claim 12, further characterized in that the ammonium sulfate concentration is between 10% to 60% by weight/volume, inclusive.

14. The method of claim 12 wherein the ammonium sulfate concentration is between 15% to 45% by weight/volume, inclusive.

15. The method according to claim 1, further characterized in that the solid support does not comprise a ligand specific for the protein of interest.

16. The method according to claim 15, further characterized in that the solid support is chosen from among organic or inorganic supports.

17. The method according to claim 16, further characterized in that the solid inorganic support is chosen from among supports comprising silica, alumina or metal oxides, and diatomaceous earth.

18. The method according to claim 16, further characterized in that the solid organic support is chosen from among supports comprising dextrans, agarose, polyacrylamide, cellulose, divinylbenzene polystyrene, nylon and methacrylate.

19. The method according to claim 1, further characterized in that it has a step of desorption of the protein of interest from the solid support, in the sustantial absence of the precipitating agent.

20. The method according to claim 19, further characterized in that it comprises one or more chromatographic purification steps.

21. The method according to claim 20, further characterized in that the chromatographic purification step is ion-exchange chromatography, size-exclusion chromatography, hydrophobic-interaction chromatography, immobilized metal-ion affinity chromatography, an affinity chromatography, or a high-performance liquid chromatography (HPLC).

22. The method according to claim 20, further characterized in that it comprises a step of passage of the protein of interest, desorbed from the solid support, onto a cation-exchange chromatographic support.

23. The method according to claim 22, further characterized in that it comprises a step of immobilized metal-ion affinity chromatography (IMAC) of the eluate from the chromatography step of claim 20.

24. The method according to claim 23, further characterized in that the chromotographic support used in IMAC chromatography comprises Cu II ions.

25. The method according to claim 20, further characterized in that said chromatography is followed by one or more ultrafiltration or dialysis steps.

26. The method according to claim 1, further characterized in that it also comprises a step of drying the purified protein by lyophilization or atomization.

27. The method according to claim 1, further characterized in that the protein of interest is initially contained in material of animal, bacterial, fungal or viral origin.

28. The method according to claim 1, further characterized in that the protein of interest is initially contained in a plant material.

29. The method according to claim 28, further characterized in that the plant material is an oleaginous, protein-containing plant and/or a plant rich in polysaccharides and/or in polyphenols and/or in pigments.

30. The method according to claim 28, further characterized in that the plant material is chosen from among corn, tobacco, tomato, canola, soy, rice, potato, carrot, wheat, barley, sunflower, lettuce or even oats.

31. The method according to claims 28, further characterized in that it comprises a first step of grinding kernels or mincing leaves, followed by a clarification step by filtration or centrifugation.

32. The method according to claim 1, further characterized in that the protein of interest is a recombinant gastric lipase.

33. The method according to claim 1, further characterized in that the protein of interest is a recombinant dog gastric lipase.

34. A recombinant dog gastric lipase purified by the method of claim 1.

35. The purified extracted or recombinant gastric lipase of claim 34, having a purity of at least 90% by weight relative to the total proteins present in the medium containing it.

36. The gastric lipase of claim 34 having a purity of at least 92% by weight relative to the total proteins present in the medium containing it.

37. The gastric lipase of claim 34 having a purity of at least 95% by weight relative to the total proteins present in the medium containing it.

38. A composition comprising a gastric lipase of claim 34.

39. A pharmaceutical composition comprising a gastric lipsase of claim 34, combined with a pharmaceutically acceptable vehicle.
